(19) United States
(12) Patent Application Publication
(10) Pub. No.: US 2023/0312477 A1
(43) Pub. Date: Oct. 5, 2023

(54) INHIBITOR OF VAPOR SPACE POLYMERIZATION

(71) Applicant: ECOLAB USA INC., St. Paul, MN (US)

(72) Inventors: Zhenxing XI, Katy, TX (US); Pedro Jorge Carvalho CAMPOS, Perre (PT); Debby ROSSANA, Tomball, TX (US); Jonathan MASERE, Richmond, TX (US)

(73) Assignee: ECOLAB USA INC., St. Paul, MN (US)

(21) Appl. No.: 18/191,507

(22) Filed: Mar. 28, 2023

Related U.S. Application Data
(60) Provisional application No. 63/326,430, filed on Apr. 1, 2022.

(51) Int. Cl. C07D 227/08; C07C 211/51

(52) U.S. Cl. CPC C07D 227/08; C07C 211/51

(57) ABSTRACT

Inhibitor compositions for abating undesirable polymerization in the vapor space during purification of reactive vinylic monomers are provided. The polymerization inhibitor compositions include at least a first inhibitor compound having a stable nitroxide radical and a second inhibitor including phenylenediamine. Methods of inhibiting the polymerization of monomers in the vapor-space using the compositions of the disclosure are also provided.

### FIELD OF THE INVENTION

The present disclosure generally relates to compositions that include a blend of polymerization inhibitors and methods of using the same to inhibit unwanted polymerization in vapor-space areas. More particularly, the present disclosure relates to compositions that include at least one compound having a stable nitroxide radical and phenylenediamine, useful for inhibiting polymerization of ethylenic unsaturated monomers in vapor-space areas.

### BACKGROUND

Ethylenically unsaturated monomers are typically manufactured in a three-stage process consisting of (i) reaction, (ii) recovery, and (iii) purification. Distillation operations at elevated temperatures are often involved in the recovery and purification stages. The processes include fractional distillation of the monomers. In the top sections of the distillation columns, purified monomer vapor condenses into liquid pools prior to transfer into storage facilities. Some of the condensed monomer streams cascade down the column. In the vapor space areas of the distillation column, the liquid monomer pools are likely to generate spontaneous or peroxide-initiated free-radicals. Current polymer inhibitor technologies are confined to liquid streams in the bottom sections of the process columns. The inhibitors are typically non-volatile so that they are confined to the bottom section of the distillation column, and no inhibitor reaches highly reactive monomer pools in the vapor-space sections. With conventional inhibitors, the pooled monomers are not treated to abate unwanted polymerization. For this reason, reactive monomers undesirably polymerize through radical polymerization especially at elevated temperature or when in contact with rust on the surfaces of the process equipment such as distillation column trays.

In the presence of polymerization initiators, such as organic peroxides, which are ubiquitous in recycled streams previously exposed to atmospheric oxygen, this polymerization is particularly acute. Conventional polymerization inhibitors, such as 4-hydroxy-2,2,6,6-tetramethylpiperidin-1-oxyl (HTEMPO) and 4-oxo-2,2,6,6-tetramethylpiperidin-1-oxyl (OTEMPO) are generally effective. The premature polymerization of these monomers is generally controlled by directly dosing polymerization inhibitors into liquid hydrocarbon streams to reduce the premature polymerization of the monomers. However, these polymerization inhibitors (HTEMPO and OTEMPO) are not volatile. In the field, the process is accelerated when liquid monomer condenses on the trays, far above the bulk liquid that is treated with non-volatile polymerization inhibitors. The problem is acute if the trays have rust or when there are residual polymers in cracks and nooks. Equally important, preexisting polymers in equipment cracks, dead legs and stagnant pools of liquid monomer cause the rapid accumulation of foulant polymer due to the Norris-Trommsdorf effect. Due to accelerated polymerization, the growing polymer foulant causes mechanical damage and potential release of vast quantities of heat resulting in fires or explosions. The complete inhibition of tray corrosion or the complete removal of polymer from hard-to-reach locations in purification towers means that preventing unwanted polymerization in vapor spaces is difficult.

### BRIEF SUMMARY

A method of inhibiting polymerization of a monomer in the vapor-space section of distillation columns is provided. The method includes adding the composition described herein to the monomer. The composition includes a first inhibitor compound comprising a stable nitroxide radical and a second inhibitor compound comprising a phenylenediamine.

In some aspects, the first inhibitor compound is of formula (I):

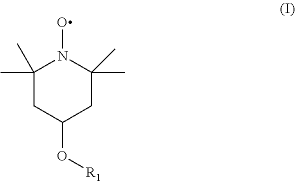

wherein R₁ is C₁-C₂₂ alkyl or aryl, wherein the alkyl and aryl are optionally substituted with one or more C₁-C₂₂ alkyl or aryl.

In some aspects, the first inhibitor is selected from the group consisting of: 1-oxyl-2,2,6,6-tetramethylpiperin-4-ol; 4-methoxy-2,2,6,6-tetramethylpiperidin-1-oxy; 4-ethoxy-2,2,6,6-tetramethylpiperidin-1-oxy; 4-propoxy-2,2,6,6-tetramethylpiperidin-1-oxy; 4-butoxy-2,2,6,6-tetramethylpiperidin-1-oxy; 4-pentoxy-2,2,6,6-tetramethylpiperidin-1-oxy; 4-hexyloxy-2,2,6,6-tetramethylpiperidin-1-oxy; 4-heptyloxy-2,2,6,6-tetramethylpiperidin-1-oxy; 4-octyloxy-2,2,6,6-tetramethylpiperidin-1-oxy; 4-nonyloxy-2,2,6,6-tetramethylpiperidin-1-oxy; 4-decyloxy-2,2,6,6-tetramethylpiperidin-1-oxy; 4-undecyloxy-2,2,6,6-tetramethylpiperidin-1-oxy; 4-dodecyloxy-2,2,6,6-tetramethylpiperidin-1-oxy; 4-tridecyloxy-2,2,6,6-tetramethylpiperidin-1-oxy; 4-tetradecyloxy-2,2,6,6-tetramethylpiperidin-1-oxy; 4-pentadecyloxy-2,2,6,6-tetramethylpiperidin-1-oxy; 4-hexadecyloxy-2,2,6,6-tetramethylpiperidin-1-oxy; 4-heptadecyloxy-2,2,6,6-tetramethylpiperidin-1-oxy; 4-octadecyloxy-2,2,6,6-tetramethylpiperidin-1-oxy; 4-nodecyloxy-2,2,6,6-tetramethylpiperidin-1-oxy; 4-decyloxy-2,2,6,6-tetramethylpiperidin-1-oxy; 4-icosyloxy-2,2,6,6-tetramethylpiperidin-1-oxy; 4-henicosyloxy-2,2,6,6-tetramethylpiperidin-1-oxy; 4-docosyloxy-2,2,6,6-tetramethylpiperidin-1-oxy; 4-(phenoxy)2,2,6,6-tetramethylpiperidin-1-oxy; 4-(benzyloxy)-2,2,6,6-tetramethylpiperidin-1-oxy; 2,2,6,6-tetramethyl-4-(naphthalen-2-yloxy)piperidin-1-oxy; and any combination thereof.

In some aspects, the first inhibitor is a compound of formula III:

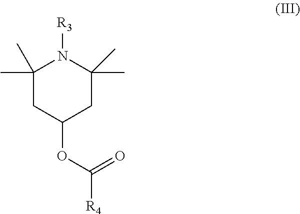

wherein R₃ is —O· or —OH; and R₄ is C₁-C₂₂ alkyl or aryl, wherein the alkyl and aryl are optionally substituted with one or more C₁-C₂₂ alkyl or aryl.

In some aspects, the first inhibitor is selected from the group consisting of: 1-oxyl-2,2,6,6-tetramethylpiperidin-4-yl acetate; 1-oxyl-2,2,6,6-tetramethylpiperidin-4-yl propanoate; 1-oxyl-2,2,6,6-tetramethylpiperidin-4-yl butyrate; 1-oxyl-2,2,6,6-tetramethylpiperidin-4-yl pentanoate; 1-oxyl-2,2,6,6-tetramethylpiperidin-4-yl hexanoate; 1-oxyl-2,2,6,6-tetramethylpiperidin-4-yl heptanoate; 1-oxyl-2,2,6,6-tetramethylpiperidin-4-yl octanoate; 1-oxyl-2,2,6,6-tetramethylpiperidin-4-yl nonanoate; 1-oxyl-2,2,6,6-tetramethylpiperidin-4-yl decanoate; 1-oxyl-2,2,6,6-tetramethylpiperidin-4-yl undecanoate; 1-oxyl-2,2,6,6-tetramethylpiperidin-4-yl dodecanoate; 1-oxyl-2,2,6,6-tetramethylpiperidin-4-yl 2-ethylhexanoate; 1-oxyl-2,2,6,6-tetramethylpiperidin-4-yl stearate; 1-oxyl-2,2,6,6-tetramethylpiperidin-4-yl benzoate; 1-oxyl-2,2,6,6-tetramethylpiperidin-4-yl palmitoate; 1-oxyl-2,2,6,6-tetramethylpiperidin-4-yl behenoate; 1-oxyl-2,2,6,6-tetramethylpiperidin-4-yl 4-tert-butylbenzoate; and any combination thereof.

In some aspects, the second inhibitor compound is a phenylenediamine of formula (IV) or formula (V):

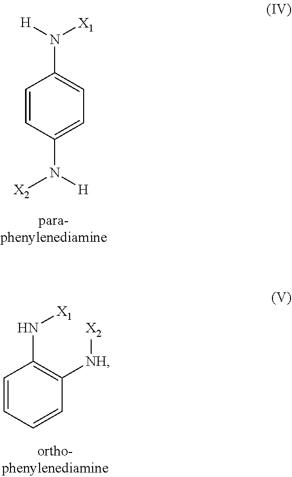

wherein X₁ and X₂ are independently C₁-C₂₂ alkyl or aryl, wherein the alkyl and aryl are optionally substituted with one or more C₁-C₂₂ alkyl or aryl.

In some aspects, X₁ and X₂ are independently C₁-C₂₂ alkyl or phenyl, wherein the alkyl and phenyl are optionally substituted with one or more C₁-C₂₂ alkyl or aryl.

In some aspects, X₁ and X₂ are independently C₁-C₁₀ alkyl or phenyl, wherein the alkyl and phenyl are optionally substituted with one or more C₁-C₂₂ alkyl or aryl.

In some aspects, X₁ and X₂ are independently C₁-C₅ alkyl or phenyl, wherein the alkyl and phenyl are optionally substituted with one or more C₁-C₂₂ alkyl or aryl.

In some aspects, the second inhibitor is selected from the group consisting of: 1,2-phenylenediamine, 1,4-phenylenediamine, N,N′-di-methyl-p-phenylenediamine, N,N′-di-sec-butyl-1,4-phenylenediamine, N,N′-di-1,4-dimethylpentyl-1,4-phenylenediamine, N, N′-di-acetyl-1,4-phenylenediamine, N-tert-butyl-N′-phenyl-1,4-phenylenediamine, N,N′-di-phenyl-1,4-phenylenediamine, and any combination thereof.

In some aspects, the first inhibitor compound is present in the composition at a concentration of about 0.01% by weight to about 80% by weight.

In some aspects, the second inhibitor compound is present in the composition at a concentration of about 0.01% by weight to about 50% by weight.

In some aspects, a mole ratio of the first inhibitor compound to the second inhibitor compound is about 100:1 to about 1:100.

In some aspects, the composition further comprises an organic solvent.

In some aspects, the composition further comprises an ethylenic unsaturated monomer selected from the group consisting of vinyl acetate, acrylonitrile, an acrylate, a methacrylate, 1,3-butadiene, styrene, isoprene, acrylic acid, methacrylic acid and any combination thereof.

A method of inhibiting monomer polymerization is provided. The method includes adding a composition to a process stream in a monomer purification process. The composition comprises a first inhibitor compound comprising a stable nitroxide radical and a second inhibitor compound comprising a phenylenediamine.

In some aspects, the process stream includes a monomer.

In some aspects, the process stream further comprises one or more additional components selected from: an acid, an organic solvent, and water.

In some aspects, the monomer is an ethylenic unsaturated monomer.

In some aspects, the composition is added to the process stream such that a concentration of the first inhibitor compound is about 0.1 ppm to about 10,000 ppm.

In some aspects, the composition is added to the process stream such that a concentration of the second inhibitor compound is about 0.1 ppm to about 10,000 ppm.

In some aspects, the monomer is selected from the group consisting of vinyl acetate, acrylonitrile, an acrylate, a methacrylate, 1,3-butadiene, styrene, divinyl benzene, isoprene, cyclopentadiene, dicyclopentadiene, acrylic acid, methacrylic acid and any combination thereof.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific aspects disclosed may be readily utilized as a basis for modifying or designing other aspects for carrying out the same purposes of the present disclosure. It should also be realized by those skilled in the art that such equivalent aspects do not depart from the spirit and scope of the disclosure as set forth in the appended claims.

### BRIEF DESCRIPTION OF THE DRAWINGS

A detailed description of the invention is hereafter described with specific reference being made to the drawings.

FIG. 1 shows vapor space polymerization inhibitor of various formulations.

### DETAILED DESCRIPTION

Various aspects of the present disclosure are described below. The relationship and functioning of the various elements of the aspects may better be understood by reference to the following detailed description. However, aspects are not limited to those explicitly described herein and it should be understood that, in certain instances, details may have been omitted that are not necessary for an understanding of the aspects disclosed herein, such as—for example—conventional synthesis and/or formulation.

During extractive distillation of reactive conjugated diene monomers, vapor space polymerization can occur leading to unwanted fouling. There is a need for polymerization inhibitors that can effectively prevent vapor space polymerization. Compositions and methods disclosed herein can prevent or reduce emulsion polymerization.

As used herein, “vapor space” refers to sections of distillation equipment typically in contact with vapor rather than a liquid process stream.

A method of inhibiting monomer polymerization is provided. The method includes adding a composition to a process stream in a monomer purification process. The composition comprises a first inhibitor compound comprising a stable nitroxide radical and a second inhibitor compound comprising a phenylenediamine.

The present disclosure relates to compositions that include a blend of polymerization inhibitors and methods of using the same to inhibit the polymerization of ethylenic unsaturated monomers. Polymerization inhibitor compositions of the present disclosure include at least one compound having a thermally and chemically stable nitroxide radical and a phenylenediamine. The polymerization inhibitor compositions can be blends of multiple components, including components in addition to the aforementioned compounds having a stable nitroxide radical and a phenylenediamine.

A “polymerization inhibitor,” in the presence of polymerizable monomers, inhibits the polymerization of these monomers during the induction time under shutdown conditions. After the induction time has elapsed following the complete consumption of the polymerization inhibitor, the polymer's formation occurs at the same rate as is the case in the total absence of the polymerization inhibitor.

Polymerization inhibitors and polymerization retarders can be considered generally as “antipolymerants” which are compounds that can inhibit or reduce the formation of polymers from one or more radically polymerizable compounds.

The term “fouling” refers to the formation of polymers, prepolymers, oligomer and/or other materials, which would become insoluble in and/or precipitate from a stream and deposit on equipment under the conditions of operation of the equipment. In turn, the inhibitor compositions of the disclosure can be referred to as “antifouling” as they inhibit or reduce the formation of foulant polymers.

### Compositions of the Disclosure

The present disclosure relates to compositions for inhibiting monomer polymerization where the compositions include a first inhibitor compound having a stable nitroxide radical and a second inhibitor compound a phenylenediamine. In some aspects, the compositions used herein are especially useful in preventing vapor space polymerization in distillation columns. In some aspects, the compositions are for inhibiting monomer polymerization, where the monomer is an ethylenic unsaturated monomer. For example, the compositions of the disclosure are useful for inhibiting polymerization of ethylenic unsaturated monomers including, but not limited to, vinyl acetate, acrylonitrile, acrylate esters, methacrylate esters, 1,3-butadiene, styrene, isoprene, acrylic acid, (meth)acrylic acid, and combinations thereof.

In some aspects, the first inhibitor compound having a stable nitroxide radical is a compound of formula (I):

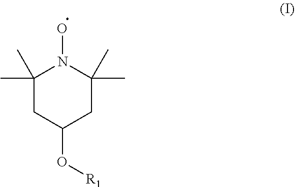

where R₁ is H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), or —C(O)(C₁-C₂₂ alkylene aryl), where the alkyl, cycloalkyl, and aryl are optionally substituted with one or more C₁-C₂₂ alkyl, C₁-C₂₂ alkenyl, C₁-C₂₂ alkynyl, or aryl.

The term “aryl” refers to monocyclic, bicyclic (fused), and tricyclic (fused or spiro) hydrocarbon ring systems having a total of five to fourteen ring carbon atoms, wherein at least one ring in the system is aromatic and wherein each ring in the system contains 3 to 7 ring carbon atoms. The term “aryl” may be used interchangeably with the term “aryl ring”.

In certain aspects, R₁ is —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), or —C(O)(C₁-C₂₂ alkylene aryl), wherein the alkyl, cycloalkyl, and aryl are optionally substituted with one or more C₁-C₂₂ alkyl, C₁-C₂₂ alkenyl, C₁-C₂₂ alkynyl, and aryl.

In some aspects, R₁ is C₁-C₂₂ alkyl or aryl, wherein the alkyl and aryl are optionally substituted with one or more C₁-C₂₂ alkyl or aryl.

In some aspects, R₁ is H. In some aspects, R₁ is C₁-C₂₂ alkyl. In some aspects, R₁ is C₁-C₂₂ alkenyl. In some aspects, R₁ is C₁-C₂₂ alkynyl. In some aspects, R₁ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some aspects, R₁ is aryl, where the aryl is optionally substituted with one or more C₁-C₂₂ alkyl, C₁-C₂₂ alkenyl, C₁-C₂₂ alkynyl, or aryl. In some aspects, R₁ is —C₁-C₂₂ alkylene aryl, where the aryl is optionally substituted with one or more C₁-C₂₂ alkyl, C₁-C₂₂ alkenyl, C₁-C₂₂ alkynyl, or aryl. In some aspects, R₁ is —C(O)(C₁-C₂₂ alkyl). In some aspects, R₁ is —C(O)(C₁-C₁₂ alkyl). In some aspects, R₁ is —C(O)(C₁-C₆ alkyl). In some aspects, R₁ is —C(O)(methyl). In some aspects, R₁ is —C(O)(ethyl). In some aspects, R₁ is —C(O)(propyl). In some aspects, R₁ is —C(O)(butyl). In some aspects, R₁ is —C(O)(C₁-C₂₂ alkenyl). In some aspects, R₁ is —C(O)(C₁-C₂₂ alkynyl). In some aspects, R₁ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some aspects, R₁ is —C(O)(aryl), where the aryl is optionally substituted with one or more C₁-C₂₂ alkyl, C₁-C₂₂ alkenyl, C₁-C₂₂ alkynyl, or aryl. In some aspects, R₁ is —C(O)(C₁-C₂₂ alkylene aryl), where the aryl is optionally substituted with one or more C₁-C₂₂ alkyl, C₁-C₂₂ alkenyl, C₁-C₂₂ alkynyl, or aryl.

Examples of compounds of formula (I) include, but are not limited to, 1-oxyl-2,2,6,6-tetramethylpiperin-4-ol; 4-methoxy-2,2,6,6-tetramethylpiperidin-1-oxy; 4-ethoxy-2,2,6,6-tetramethylpiperidin-1-oxy; 4-propoxy-2,2,6,6-tetramethylpiperidin-1-oxy; 4-butoxy-2,2,6,6-tetramethylpiperidin-1-oxy; 4-pentoxy-2,2,6,6-tetramethylpiperidin-1-oxy; 4-hexyloxy-2,2,6,6-tetramethylpiperidin-1-oxy; 4-heptyloxy-2,2,6,6-tetramethylpiperidin-1-oxy; 4-octyloxy-2,2,6,6-tetramethylpiperidin-1-oxy; 4-nonyloxy-2,2,6,6-tetramethylpiperidin-1-oxy; 4-decyloxy-2,2,6,6-tetramethylpiperidin-1-oxy; 4-undecyloxy-2,2,6,6-tetramethylpiperidin-1-oxy; 4-dodecyloxy-2,2,6,6-tetramethylpiperidin-1-oxy; 4-tridecyloxy-2,2,6,6-tetramethylpiperidin-1-oxy; 4-tetradecyloxy-2,2,6,6-tetramethylpiperidin-1-oxy; 4-pentadecyloxy-2,2,6,6-tetramethylpiperidin-1-oxy; 4-hexadecyloxy-2,2,6,6-tetramethylpiperidin-1-oxy; 4-heptadecyloxy-2,2,6,6-tetramethylpiperidin-1-oxy; 4-octadecyloxy-2,2,6,6-tetramethylpiperidin-1-oxy; 4-nodecyloxy-2,2,6,6-tetramethylpiperidin-1-oxy; 4-decyloxy-2,2,6,6-tetramethylpiperidin-1-oxy; 4-icosyloxy-2,2,6,6-tetramethylpiperidin-1-oxy; 4-henicosyloxy-2,2,6,6-tetramethylpiperidin-1-oxy; 4-docosyloxy-2,2,6,6-tetramethylpiperidin-1-oxy; 4-(phenoxy)2,2,6,6-tetramethylpiperidin-1-oxy; 4-(benzyloxy)-2,2,6,6-tetramethylpiperidin-1-oxy; or 2,2,6,6-tetramethyl-4-(naphthalen-2-yloxy)piperidin-1-oxy.

In other aspects, the first inhibitor compound is of formula (II):

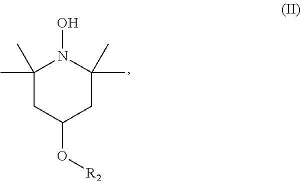

where R₂ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene), wherein the alkyl, alkylene, cycloalkyl, and aryl are optionally substituted with one or more C₁-C₂₂ alkyl, C₁-C₂₂ alkenyl, C₁-C₂₂ alkynyl, or aryl.

In certain aspects, R₂ is —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene), where the alkyl, alkylene, cycloalkyl, and aryl are optionally substituted with one or more C₁-C₂₂ alkyl, C₁-C₂₂ alkenyl, C₁-C₂₂ alkynyl, or aryl.

In some aspects, R₂ is H. In some aspects, R₂ is C₁-C₂₂ alkyl. In some aspects, R₂ is C₁-C₂₂ alkenyl. In some aspects, R₂ is C₁-C₂₂ alkynyl. In some aspects, R₂ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some aspects, R₂ is aryl, where the aryl is optionally substituted with one or more C₁-C₂₂ alkyl, C₁-C₂₂ alkenyl, C₁-C₂₂ alkynyl, or aryl. In some aspects, R₂ is —C₁-C₂₂ alkylene, where the alkylene is optionally substituted with aryl that is optionally substituted with one or more C₁-C₂₂ alkyl, C₁-C₂₂ alkenyl, C₁-C₂₂ alkynyl, or aryl. In some aspects, R₂ is —C(O)(C₁-C₂₂ alkyl). In some aspects, R₂ is —C(O)(C₁-C₁₂ alkyl). In some aspects, R₂ is —C(O)(C₁-C₆ alkyl). In some aspects, R₂ is —C(O)(methyl). In some aspects, R₂ is —C(O)(ethyl). In some aspects, R₂ is —C(O)(propyl). In some aspects, R₂ is —C(O)(butyl). In some aspects, R₂ is —C(O)(C₁-C₂₂ alkenyl). In some aspects, R₂ is —C(O)(C₁-C₂₂ alkynyl). In some aspects, R₂ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some aspects, R₂ is —C(O)(aryl), where the aryl is optionally substituted with one or more C₁-C₂₂ alkyl, C₁-C₂₂ alkenyl, C₁-C₂₂ alkynyl, or aryl. In some aspects, R₂ is —C(O)(C₁-C₂₂ alkylene), where the alkylene is optionally substituted with aryl that is optionally substituted with one or more C₁-C₂₂ alkyl, C₁-C₂₂ alkenyl, C₁-C₂₂ alkynyl, or aryl.

In some aspects, the compound of formula (II) is 2,2,6,6-tetramethylpiperin-1,4-diol; 4-methoxy-2,2,6,6-tetramethylpiperidin-1-ol; 4-ethoxy-2,2,6,6-tetramethylpiperidin-1-ol; 4-propoxy-2,2,6,6-tetramethylpiperidin-1-ol; 4-butoxy-2,2,6,6-tetramethylpiperidin-1-ol; 4-pentoxy-2,2,6,6-tetramethylpiperidin-1-ol; 4-hexyloxy-2,2,6,6-tetramethylpiperidin-1-ol; 4-heptyloxy-2,2,6,6-tetramethylpiperidin-1-ol; 4-octyloxy-2,2,6,6-tetramethylpiperidin-1-ol; 4-nonyloxy-2,2,6,6-tetramethylpiperidin-1-ol; 4-decyloxy-2,2,6,6-tetramethylpiperidin-1-ol; 4-undecyloxy-2,2,6,6-tetramethylpiperidin-1-ol; 4-dodecyloxy-2,2,6,6-tetramethylpiperidin-1-ol; 4-tridecyloxy-2,2,6,6-tetramethylpiperidin-1-ol; 4-tetradecyloxy-2,2,6,6-tetramethylpiperidin-1-ol; 4-pentadecyloxy-2,2,6,6-tetramethylpiperidin-1-ol; 4-hexadecyloxy-2,2,6,6-tetramethylpiperidin-1-ol; 4-heptadecyloxy-2,2,6,6-tetramethylpiperidin-1-ol; 4-octadecyloxy-2,2,6,6-tetramethylpiperidin-1-ol; 4-nodecyloxy-2,2,6,6-tetramethylpiperidin-1-ol; 4-decyloxy-2,2,6,6-tetramethylpiperidin-1-ol; 4-icosyloxy-2,2,6,6-tetramethylpiperidin-1-ol; 4-henicosyloxy-2,2,6,6-tetramethylpiperidin-1-ol; 4-docosyloxy-2,2,6,6-tetramethylpiperidin-1-ol; 4-(phenoxy)-2,2,6,6-tetramethylpiperidin-1-ol; 4-(benzyloxy)-2,2,6,6-tetramethylpiperidin-1-ol; or 2,2,6,6-tetramethyl-4-(naphthalen-2-yloxy)piperidin-1-ol.

In certain aspects, the compositions of the disclosure include compounds of formula (I) and (II), respectively, where R₁ and R₂ are the same. For example, in some aspects, the compositions of the disclosure include compounds of formula (I) and (II), respectively, where R₁ and R₂ are each, independently, —C(O)(C₁-C₂₂ alkyl). In certain aspects, the compositions of the disclosure include first and second inhibitor compounds of formula (I) and (II), respectively, where R₁ and R₂ are different.

The presently disclosed compound of formula (II) having a hydroxylamine has benefits over the corresponding nitroxide (compound of formula (I)), such as the capability to provide additional polymerization inhibition, as will be more fully explained below. A general synthetic route to produce a hydroxylamine of a nitroxide is to reduce its corresponding nitroxide with a reducing reagent as follows:

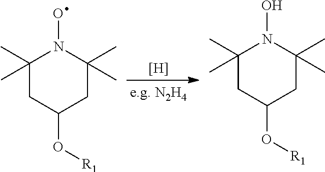

A hydroxylamine of a nitroxide has the potential to provide additional polymerization inhibition as compared to the corresponding nitroxide when carbon-centered and oxygen-centered radical initiators are present. This is explained as follows:

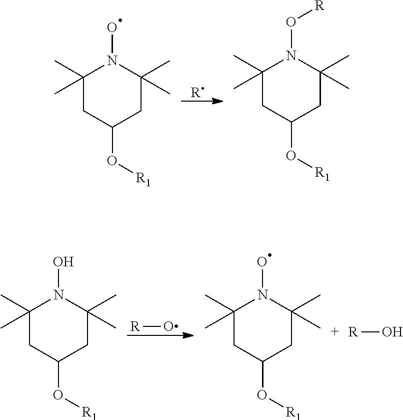

The hydroxylamine of a nitroxide is an excellent hydrogen donor due to its weak NO—H bond in the compound, and thus it is an efficient antioxidant. As an antioxidant, the hydroxylamine of a nitroxide easily reacts with oxygen-centered radicals, such as peroxide radicals, while it's converted to its corresponding nitroxide. Nitroxides are generally known as the most effective inhibitors because of their superior inhibiting capabilities through scavenging carbon-centered free radicals at a nearly diffusion controlled rate. This rate is several orders of magnitude faster than phenolic compounds. However, their kinetic superiority is not always advantageous. For instance, it may lose its superiority when oxygen-centered radicals are present as the predominant free radicals. Another issue of concern with a nitroxide is its consumption through non-inhibition and unwanted reactions with process stream components or other inhibitor additives. As a result, high nitroxide inhibitor dosages are often required for a given inhibition efficacy, thereby making their use economically unattractive or even infeasible.

In essence, each hydroxylamine of a nitroxide is equivalent to one hydrogen donor plus one nitroxide antipolymerant when oxygen-centered radicals and carbon-centered radicals are both present, which is an attractive incentive offered by the hydroxylamines of nitroxides. That is, one hydroxylamine of a nitroxide is able to eliminate one oxygen-centered radical and one carbon-centered radical whereas a nitroxide is only capable to eliminate a carbon-centered radical.

In some aspects, the first inhibitor is a compound of formula III:

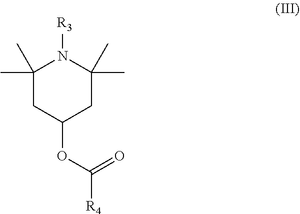

wherein R₃ is —O·0 or —OH; and R₄ is C₁-C₂₂ alkyl or aryl, wherein the alkyl and aryl are optionally substituted with one or more C₁-C₂₂ alkyl or aryl.

In some aspects, R₃ is —O·. In some aspects, R₃ is —OH.

In some aspects, R₄ is C₁-C₂₂ alkyl that is optionally substituted with one or more C₁-C₂₂ alkyl or aryl. In some aspects, R₄ is aryl that is optionally substituted with one or more C₁-C₂₂ alkyl or aryl.

Examples of a compound of formula (III) include, but are not limited to, 1-oxyl-2,2,6,6-tetramethylpiperidin-4-yl acetate; 1-oxyl-2,2,6,6-tetramethylpiperidin-4-yl propanoate; 1-oxyl-2,2,6,6-tetramethylpiperidin-4-yl butyrate; 1-oxyl-2,2,6,6-tetramethylpiperidin-4-yl pentanoate; 1-oxyl-2,2,6,6-tetramethylpiperidin-4-yl hexanoate; 1-oxyl-2,2,6,6-tetramethylpiperidin-4-yl heptanoate; 1-oxyl-2,2,6,6-tetramethylpiperidin-4-yl octanoate; 1-oxyl-2,2,6,6-tetramethylpiperidin-4-yl nonanoate; 1-oxyl-2,2,6,6-tetramethylpiperidin-4-yl decanoate; 1-oxyl-2,2,6,6-tetramethylpiperidin-4-yl undecanoate; 1-oxyl-2,2,6,6-tetramethylpiperidin-4-yl dodecanoate; 1-oxyl-2,2,6,6-tetramethylpiperidin-4-yl 2-ethylhexanoate; 1-oxyl-2,2,6,6-tetramethylpiperidin-4-yl stearate; 1-oxyl-2,2,6,6-tetramethylpiperidin-4-yl benzoate; 1-oxyl-2,2,6,6-tetramethylpiperidin-4-yl palmitoate; 1-oxyl-2,2,6,6-tetramethylpiperidin-4-yl behenoate; or 1-oxyl-2,2,6,6-tetramethylpiperidin-4-yl 4-tert-butylbenzoate.

In some aspects, the second inhibitor compound is a phenylenediamine of formula (IV) or formula (V):

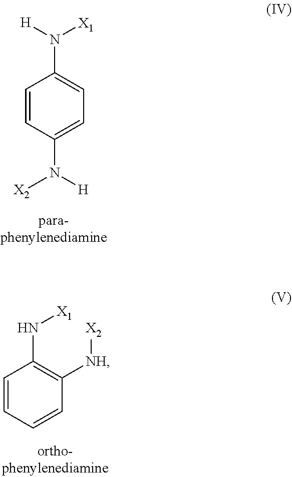

wherein X₁ and X₂ are independently C₁-C₂₂ alkyl, or aryl, wherein the alkyl and aryl are optionally substituted with one or more C₁-C₂₂ alkyl or aryl.

In some aspects, the second inhibitor compound is a phenylenediamine of formula (IV). In some aspects, the second inhibitor compound is a phenylenediamine of formula (V).

Examples of phenylenediamines include, but are not limited to, 1,2-phenylenediamine, 1,4-phenylenediamine, N,N′-di-methyl-p-phenylenediamine, N,N′-di-sec-butyl-1,4-phenylenediamine, N,N′-di-1,4-dimethylpentyl-1,4-phenylenediamine, N, N′-di-acetyl-1,4-phenylenediamine, N-tert-butyl-N′-phenyl-1,4-phenylenediamine, and N,N′-di-phenyl-1,4-phenylenediamine.

In some aspects, the composition includes 2,2′,6,6′-tetramethylpiperidinyl-1-oxyl and an alkyl substituted 1,4-phenylenediamine.

In some aspects, the composition consists essentially of a first inhibitor compound and a second inhibitor compound. In other aspects, the composition consists of an organic solvent, a first inhibitor, and a second inhibitor.

In some aspects, the first inhibitor compound is present in the composition at a concentration of about 0.01% by weight to about 80% by weight. In some aspects, the first inhibitor compound is present in the composition at a concentration of about 0.01% by weight to about 70% by weight. In some aspects, the first inhibitor compound is present in the composition at a concentration of about 0.01% by weight to about 60% by weight. In some aspects, the first inhibitor compound is present in the composition at a concentration of about 0.01% by weight to about 50% by weight. In some aspects, the first inhibitor compound is present in the composition at a concentration of about 0.01% by weight to about 40% by weight. In some aspects, the first inhibitor compound is present in the composition at a concentration of about 0.01% by weight to about 30% by weight. In some aspects, the first inhibitor compound is present in the composition at a concentration of about 0.01% by weight to about 20% by weight. In some aspects, the first inhibitor compound is present in the composition at a concentration of about 0.01% by weight to about 10% by weight.

For example, in certain aspects, the first inhibitor compound is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, or about 80% by weight.

In some aspects, the second inhibitor compound is present in the composition at a concentration of about 0.01% by weight to about 50% by weight. In some aspects, the second inhibitor compound is present in the composition at a concentration of about 0.01% by weight to about 40% by weight. In some aspects, the second inhibitor compound is present in the composition at a concentration of about 0.01% by weight to about 30% by weight. In some aspects, the second inhibitor compound is present in the composition at a concentration of about 0.01% by weight to about 20% by weight. In some aspects, the second inhibitor compound is present in the composition at a concentration of about 0.01% by weight to about 10% by weight.

For example, in certain aspects, the second inhibitor compound is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, or about 50% by weight.

In some aspects, a mole ratio of the first inhibitor compound to the second inhibitor compound is about 100:1 to about 1:100. In some aspects, a mole ratio of the first inhibitor compound to the second inhibitor compound is about 90:1 to about 1:90. In some aspects, a mole ratio of the first inhibitor compound to the second inhibitor compound is about 80:1 to about 1:80. In some aspects, a mole ratio of the first inhibitor compound to the second inhibitor compound is about 70:1 to about 1:70. In some aspects, a mole ratio of the first inhibitor compound to the second inhibitor compound is about 60:1 to about 1:60. In some aspects, a mole ratio of the first inhibitor compound to the second inhibitor compound is about 50:1 to about 1:50. In some aspects, a mole ratio of the first inhibitor compound to the second inhibitor compound is about 40:1 to about 1:40. In some aspects, a mole ratio of the first inhibitor compound to the second inhibitor compound is about 30:1 to about 1:30. In some aspects, a mole ratio of the first inhibitor compound to the second inhibitor compound is about 20:1 to about 1:20. In some aspects, a mole ratio of the first inhibitor compound to the second inhibitor compound is about 10:1 to about 1:10. In some aspects, a mole ratio of the first inhibitor compound to the second inhibitor compound is about 1:1.

In some aspects, the composition also includes one or more additional compounds selected from the group consisting of 2,2,6,6-tetramethylpiperidin-1-oxyl; 2,2,6,6-tetramethylpiperidin-1-ol; 4-hydroxyl-2,2,6,6-tetramethylpiperidin-1-oxyl; 4-hydroxy-2,2,6,6-tetramethylpiperidin-1-ol; 4-oxo-2,2,6,6-tetramethylpiperidin-1-oxyl; 4-oxo-2,2,6,6-tetramethylpiperidin-1-ol; 4-acetoxy-2,2,6,6-tetramethylpiperidin-1-oxyl; 4-acetoxy-2,2,6,6-tetramethylpiperidin-1-ol; 4-propionoxy-2,2,6,6-tetramethylpiperidin-1-oxyl; 4-propionoxy-2,2,6,6-tetramethylpiperidin-1-ol; and bis((2,2,6,6-tetramethylpiperidin-1-oxyl)-4-yl) oxalate. In some aspects, the composition also includes 2,2,6,6-tetramethylpiperidin-1-oxyl. In some aspects, the composition also includes 2,2,6,6-tetramethylpiperidin-1-ol. In some aspects, the composition also includes 4-hydroxyl-2,2,6,6-tetramethylpiperidin-1-oxyl. In some aspects, the composition also includes 4-hydroxy-2,2,6,6-tetramethylpiperidin-1-ol. In some aspects, the composition also includes 4-oxo-2,2,6,6-tetramethylpiperidin-1-oxyl. In some aspects, the composition also includes 4-oxo-2,2,6,6-tetramethylpiperidin-1-ol. In some aspects, the composition also includes 4-acetoxy-2,2,6,6-tetramethylpiperidin-1-oxyl. In some aspects, the composition also includes 4-acetoxy-2,2,6,6-tetramethylpiperidin-1-ol. In some aspects, the composition also includes 4-propionoxy-2,2,6,6-tetramethylpiperidin-1-oxyl. In some aspects, the composition also includes 4-propionoxy-2,2,6,6-tetramethylpiperidin-1-ol. In some aspects, the composition also includes bis((2,2,6,6-tetramethylpiperidin-1-oxyl)-4-yl) oxalate.

The composition may optionally also include one or more organic solvents. One of ordinary skill in the art will appreciate that there are many organic solvents that are compatible with the compositions of the disclosure. For example, in some aspects, the one or more organic solvents are selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In certain aspects, the composition also includes vinyl acetate. In certain aspects, the composition also includes dimethyl phthalate. In certain aspects, the composition also includes dimethylformamide. In certain aspects, the composition also includes toluene. In certain aspects, the composition also includes xylene. In certain aspects, the composition also includes highly aromatic naphtha. In certain aspects, the composition also includes acetonitrile.

In some aspects, the composition also includes one or more ethylenic unsaturated monomers. One of ordinary skill in the art will appreciate that there are many ethylenic unsaturated monomers that are compatible with the compositions of the disclosure. For example, in some aspects, the one or more ethylenic unsaturated monomers are selected from vinyl acetate, acrylonitrile, acrylates, methacrylates, 1,3-butadiene, styrene, isoprene, (meth)acrylic acid, and combinations thereof. In certain aspects, the composition also includes vinyl acetate. In certain aspects, the composition also includes acrylonitrile. In certain aspects, the composition also includes acrylates. In certain aspects, the composition also includes methacrylates. In certain aspects, the composition also includes 1,3-butadiene. In certain aspects, the composition also includes styrene. In certain aspects, the composition also includes isoprene. In certain aspects, the composition also includes (meth)acrylic acid.

The compositions of the disclosure are stable and remain useful polymerization inhibitors even under acidic conditions. Thus, the compositions of the disclosure are useful for inhibiting the premature polymerization of monomers during manufacturing process, particularly those that are performed under acidic conditions. For example, the compositions of the disclosure are useful for preventing polymerization of acrylates, which may include, but are not limited to, acrylonitrile, acrylic acid, methyl methacrylic acid and its esters, and vinyl acetate.

In some aspects, the composition disclosed herein do not include 4-hydroxy-2,2,6,6-tetramethylpiperidin-1-oxyl. In some aspects, the composition disclosed herein do not include 4-oxo-2,2,6,6-tetramethylpiperidin-1-oxyl. In some aspects, the 4-hydroxy-2,2,6,6-tetramethylpiperidin-1-oxyl and 4-oxo-2,2,6,6-tetramethylpiperidin-1-oxyl are not added to ethylenic unsaturated monomers.

The present disclosure also relates to methods of inhibiting polymerization of monomers that include adding a composition of the disclosure to the monomer. In some aspects, an effective amount of the composition of the disclosure is added to the monomer, where an effective amount is any amount sufficient to inhibit the polymerization of the monomer.

In some aspects, the monomer is an ethylenic unsaturated monomer. In some aspects the monomer is an ethylenic unsaturated monomer selected from vinyl acetate, acrylonitrile, acrylate esters, methacrylate esters, 1,3-butadiene, styrene, divinyl benzene, isoprene, cyclopentadiene, dicyclopentadiene, acrylic acid, (meth)acrylic acid, and combinations thereof are disclosed. In some aspects, the methods disclosed herein are useful in inhibiting the polymerization of vinyl acetate. In some aspects, the methods disclosed herein are useful in inhibiting the polymerization of acrylonitrile. In some aspects, the methods disclosed herein are useful in inhibiting the polymerization of acrylate esters. In some aspects, the methods disclosed herein are useful in inhibiting the polymerization of methacrylate esters. In some aspects, the methods disclosed herein are useful in inhibiting the polymerization of 1,3-butadiene. In some aspects, the methods disclosed herein are useful in inhibiting the polymerization of styrene. In some aspects, the methods disclosed herein are useful in inhibiting the polymerization of isoprene. In some aspects, the methods disclosed herein are useful in inhibiting the polymerization of acrylic acid. In some aspects, the methods disclosed herein are useful in inhibiting the polymerization of (meth)acrylic acid.

The composition of the disclosure can be added manually or automatically to the fluid. The composition can also be added continuously and/or intermittently. Automatic addition may be accomplished through the use of chemical injection pumps. The chemical injection pumps may be programmed to add particular amounts of the polymerization inhibitor composition, or any components thereof, at certain time intervals to the fluid. In alternate aspects, the chemical injection pumps can be manually controlled to add particular amounts of the polymerization inhibitor composition, or any components thereof, to the fluid. Addition of the presently disclosed polymerization inhibitor compositions to the monomer will thereby inhibit polymerization of the monomer.

In some aspects, the monomer is provided as a neat liquid. In other aspects, the monomer is provided within a solution, hereafter referred to as “the monomer solution.”

In some aspects, the monomer solution also includes one or more additional components selected from an acid, an organic solvent, water, and combinations thereof. For example, in some aspects, the monomer solution includes one or more organic solvents selected from vinyl acetate, dimethyl phthalate, dimethylformamide, toluene, ethyltoluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In some aspects, the monomer solution includes one or more acids selected from hydrochloric acid, hydrofluoric acid, hydrobromic acid, hydroiodic acid, nitric acid, phosphoric acid, sulfuric acid, boric acid, perchloric acid, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, and suberic acid. In some aspects, the monomer solution includes water.

In some aspects, the monomer solution has a pH value of about 1 to about 7. In some aspects, the monomer solution has a pH value of about 1 to about 6. In some aspects, the monomer solution has a pH value of about 2 to about 6. In some aspects, the monomer solution has a pH value of about 3 to about 6. In some aspects, the monomer solution has a pH value of about 4 to about 6. In some aspects, the monomer solution has a pH value of about 5 to about 6.

In some aspects, the composition is added to the monomer such that a concentration of the first inhibitor compound is about 0.1 ppm to about 10,000 ppm. In some aspects, the composition is added to the monomer such that a concentration of the first inhibitor compound is about 0.1 ppm to about 5,000 ppm. In some aspects, the composition is added to the monomer such that a concentration of the first inhibitor compound is about 0.1 ppm to about 1,000 ppm. In some aspects, the composition is added to the monomer such that a concentration of the first inhibitor compound is about 0.1 ppm to about 500 ppm.

In some aspects, the composition is added to the monomer such that a concentration of the second inhibitor compound is about 0.1 ppm to about 10,000 ppm. In some aspects, the composition is added to the monomer such that a concentration of the second inhibitor compound is about 0.1 ppm to about 5,000 ppm. In some aspects, the composition is added to the monomer such that a concentration of the second inhibitor compound is about 0.1 ppm to about 1,000 ppm. In some aspects, the composition is added to the monomer such that a concentration of the second inhibitor compound is about 0.1 ppm to about 500 ppm.

The methods of the disclosure are useful for inhibiting the premature polymerization of monomers during the extraction process. During the extraction process, solvents are used to separate components. The difference in polarity between the extraction solvents and the hydrocarbon layer results in emulsion formation, thereby increasing the risk of emulsion polymerization.

The methods of the disclosure are also useful for preventing the premature polymerization of styrene during manufacturing and purification processes.

The methods of the disclosure are also useful in butadiene extraction processes. This utility stems from the balanced partition coefficients between polar organic phases and organic phases.

In some aspects, the compositions disclosed herein are used in distillative purification of olefins. For example, the composition can be added to the process stream before entering the distillation unit or the composition can be added to the process stream in the distillation unit.

### EXAMPLES

### Example 1

Various formulations were screened for vapor space polymerization inhibition. It was discovered that a volatile nitroxide free radical and alkyl substituted 1,4-phenylenediamine exhibits synergistic effect and is superior to commercialized vapor space polymerization inhibitor such as dialkylhydroxylamine and nitroxide free radical HTEMPO.

Isoprene was passed through an alumina column to remove 4-tert-butylcatechol (TBC). About 100 ppm of active inhibitor was added to TBC free isoprene (around 125 g). The solution was purged with nitrogen. Several popcorn polymer seeds weighing around 0.15 g were added in the top of sample kit. The tubes were sealed and heated to 80° C. with a regulated stir rate of 250 rpm for 3 days. The weight gained in the kits reflected the inhibitor performance toward popcorn type polymer formation.

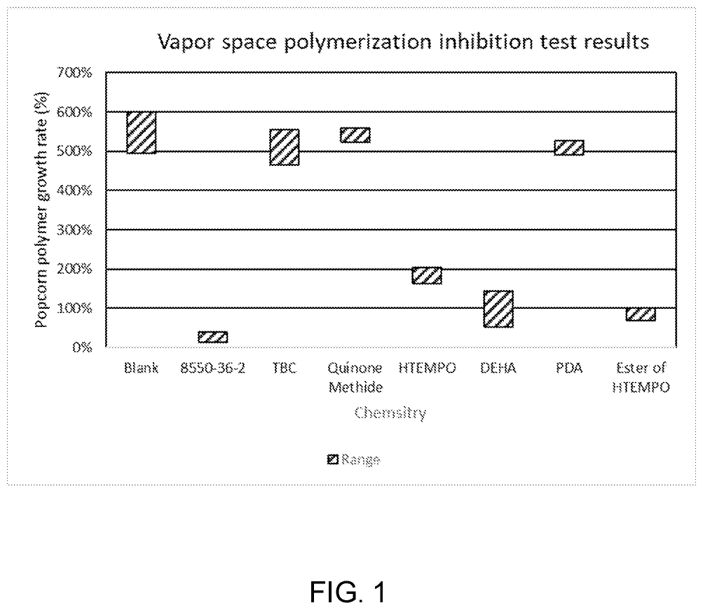

FIG. 1 shows that the combination of a volatile nitroxide free radical and alkyl substituted 1,4-phenylenediamine exhibited a synergistic effect. In FIG. 1, Ester of HTEMPO refers to 4-acetoxyl-2,2′6,6′-tetramethylpiperidinyl-1-oxyl; 8550-36-2 refers to 15 wt % 4-acetoxyl-2,2′6,6′-tetramethylpiperidinyl-1-hydoxyl and 5 wt % di-sec-butyl-4,4′-phenylenediamine; DEHA is N,N′-diethylhydroxylamine; and PDA is di-sec-butyl-4,4′-phenylenediamine.

All of the compositions and methods disclosed and claimed herein can be made and executed without undue experimentation in light of the present disclosure. While this invention may be embodied in many different forms, there are described in detail herein specific preferred aspects of the invention. The present disclosure is an exemplification of the principles of the invention and is not intended to limit the invention to the particular aspects illustrated. In addition, unless expressly stated to the contrary, use of the term “a” or “an” is intended to include “at least one” or “one or more.” For example, “an inhibitor” is intended to include “at least one inhibitor” or “one or more inhibitors.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various aspects described herein. It should also be understood that various changes and modifications to the presently preferred aspects described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of inhibiting vapor-space monomer polymerization, comprising:
adding a composition to a process stream in a monomer purification process,
wherein the composition comprises a first inhibitor compound comprising a stable nitroxide radical; and a second inhibitor compound comprising a phenylenediamine.

2. The method of claim 1, wherein the first inhibitor compound is of formula (I): 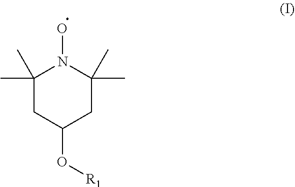
wherein R₁ is C₁-C₂₂ alkyl or aryl, wherein the alkyl and aryl are optionally substituted with one or more C₁-C₂₂ alkyl or aryl.

3. The method of claim 1, wherein the first inhibitor is selected from the group consisting of: 1-oxyl-2,2,6,6-tetramethylpiperin-4-ol; 4-methoxy-2,2,6,6-tetramethylpiperidin-1-oxy; 4-ethoxy-2,2,6,6-tetramethylpiperidin-1-oxy; 4-propoxy-2,2,6,6-tetramethylpiperidin-1-oxy; 4-butoxy-2,2,6,6-tetramethylpiperidin-1-oxy; 4-pentoxy-2,2,6,6-tetramethylpiperidin-1-oxy; 4-hexyloxy-2,2,6,6-tetramethylpiperidin-1-oxy; 4-heptyloxy-2,2,6,6-tetramethylpiperidin-1-oxy; 4-octyloxy-2,2,6,6-tetramethylpiperidin-1-oxy; 4-nonyloxy-2,2,6,6-tetramethylpiperidin-1-oxy; 4-decyloxy-2,2,6,6-tetramethylpiperidin-1-oxy; 4-undecyloxy-2,2,6,6-tetramethylpiperidin-1-oxy; 4-dodecyloxy-2,2,6,6-tetramethylpiperidin-1-oxy; 4-tridecyloxy-2,2,6,6-tetramethylpiperidin-1-oxy; 4-tetradecyloxy-2,2,6,6-tetramethylpiperidin-1-oxy; 4-pentadecyloxy-2,2,6,6-tetramethylpiperidin-1-oxy; 4-hexadecyloxy-2,2,6,6-tetramethylpiperidin-1-oxy; 4-heptadecyloxy-2,2,6,6-tetramethylpiperidin-1-oxy; 4-octadecyloxy-2,2,6,6-tetramethylpiperidin-1-oxy; 4-nodecyloxy-2,2,6,6-tetramethylpiperidin-1-oxy; 4-decyloxy-2,2,6,6-tetramethylpiperidin-1-oxy; 4-icosyloxy-2,2,6,6-tetramethylpiperidin-1-oxy; 4-henicosyloxy-2,2,6,6-tetramethylpiperidin-1-oxy; 4-docosyloxy-2,2,6,6-tetramethylpiperidin-1-oxy; 4-(phenoxy)2,2,6,6-tetramethylpiperidin-1-oxy; 4-(benzyloxy)-2,2,6,6-tetramethylpiperidin-1-oxy; 2,2,6,6-tetramethyl-4-(naphthalen-2-yloxy)piperidin-1-oxy; and any combination thereof.

4. The method of claim 1, wherein the first inhibitor is a compound of formula III: 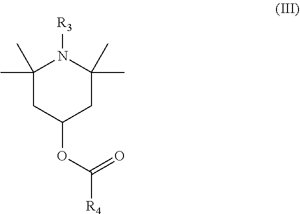
wherein R₃ is —O· or —OH; and R₄ is C₁-C₂₂ alkyl or aryl, wherein the alkyl and aryl are optionally substituted with one or more C₁-C₂₂ alkyl or aryl.

5. The method of claim 1, wherein the first inhibitor is selected from the group consisting of: 1-oxyl-2,2,6,6-tetramethylpiperidin-4-yl acetate; 1-oxyl-2,2,6,6-tetramethylpiperidin-4-yl propanoate; 1-oxyl-2,2,6,6-tetramethylpiperidin-4-yl butyrate; 1-oxyl-2,2,6,6-tetramethylpiperidin-4-yl pentanoate; 1-oxyl-2,2,6,6-tetramethylpiperidin-4-yl hexanoate; 1-oxyl-2,2,6,6-tetramethylpiperidin-4-yl heptanoate; 1-oxyl-2,2,6,6-tetramethylpiperidin-4-yl octanoate; 1-oxyl-2,2,6,6-tetramethylpiperidin-4-yl nonanoate; 1-oxyl-2,2,6,6-tetramethylpiperidin-4-yl decanoate; 1-oxyl-2,2,6,6-tetramethylpiperidin-4-yl undecanoate; 1-oxyl-2,2,6,6-tetramethylpiperidin-4-yl dodecanoate; 1-oxyl-2,2,6,6-tetramethylpiperidin-4-yl 2-ethylhexanoate; 1-oxyl-2,2,6,6-tetramethylpiperidin-4-yl stearate; 1-oxyl-2,2,6,6-tetramethylpiperidin-4-yl benzoate; 1-oxyl-2,2,6,6-tetramethylpiperidin-4-yl palmitoate; 1-oxyl-2,2,6,6-tetramethylpiperidin-4-yl behenoate; 1-oxyl-2,2,6,6-tetramethylpiperidin-4-yl 4-tert-butylbenzoate; and any combination thereof.

6. The method of claim 1, wherein the second inhibitor compound is a phenylenediamine of formula (IV) or formula (V): 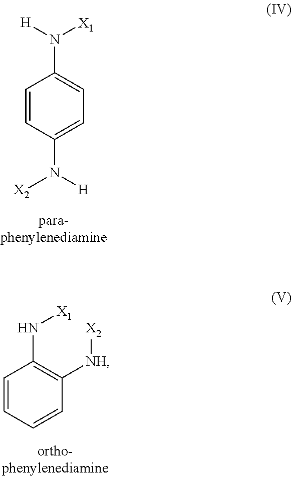
wherein X₁ and X₂ are independently C₁-C₂₂ alkyl, or aryl, wherein the alkyl and aryl are optionally substituted with one or more C₁-C₂₂ alkyl or aryl.

7. The method of claim 1, wherein the second inhibitor is selected from the group consisting of: 1,2-phenylenediamine, 1,4-phenylenediamine, N,N′-di-methyl-p-phenylenediamine, N,N′-di-sec-butyl-1,4-phenylenediamine, N,N′-di-1,4-dimethylpentyl-1,4-phenylenediamine, N, N′-di-acetyl-1,4-phenylenediamine, N-tert-butyl-N′-phenyl-1,4-phenylenediamine, N,N′-di-phenyl-1,4-phenylenediamine, and any combination thereof.

8. The method of claim 1, wherein the first inhibitor compound is present in the composition at a concentration of about 0.01% by weight to about 80% by weight.

9. The method of claim 1, wherein the second inhibitor compound is present in the composition at a concentration of about 0.01% by weight to about 50% by weight.

10. The method of claim 1, wherein a mole ratio of the first inhibitor compound to the second inhibitor compound is about 100:1 to about 1:100.

11. The method of claim 1, wherein the composition further comprises an organic solvent.

12. The method of claim 1, wherein the process stream further comprises an ethylenic unsaturated monomer selected from the group consisting of vinyl acetate, acrylonitrile, an acrylate, a methacrylate, 1,3-butadiene, styrene, isoprene, cyclopentadiene, dicyclopentadiene, acrylic acid, methacrylic acid and any combination thereof.

13. The method of claim 1, wherein the process stream further comprises one or more additional components selected from: an acid, an organic solvent, and water.

14. The method of claim 1, wherein the composition is added to the process stream such that a concentration of the first inhibitor compound is about 0.1 ppm to about 10,000 ppm.

15. The method of claim 1, wherein the composition is added to the process stream such that a concentration of the second inhibitor compound is about 0.1 ppm to about 10,000 ppm.
